(19) United States
(12) Patent Application Publication
(10) Pub. No.: US 2024/0150281 A1
(43) Pub. Date: May 9, 2024

(54) PROCESS FOR PREPARING A THIOETHER

(71) Applicant: Firmenich SA, Satigny (CH)

(72) Inventors: Gilles ODDON, Satigny (CH); Erik FREROT, Satigny (CH); Ramez BOSHRA, Newark, NJ (US)

(21) Appl. No.: 18/546,239

(22) PCT Filed: Feb. 8, 2022
(86) PCT No.: PCT/EP2022/053012

§ 371 Date: Aug. 11, 2023

Related U.S. Application Data
(60) Provisional application No. 63/148,182, filed on Feb. 11, 2021.

(30) Foreign Application Priority Data
Feb. 22, 2021 (EP) 21158321.6

(51) Int. Cl. C07C 323/22; G01N 30/72

(52) U.S. Cl. CPC C07C 323/22; G01N 30/7206; G01N 2030/884

(57) ABSTRACT

The present invention relates to the field of organic synthesis and more specifically it concerns a process for preparing a compound of formula (I). Moreover, the present invention comprises also analytical methods of measuring the amount of thiol or thioether compounds having less than 6 carbon atoms in the thiol of formula R—SH and a composition of matter comprising at most 50 μg/kg of thiol or thioether compounds having less than 6 carbon atoms and at most 1 ppm of any compound of formula (I′).

### TECHNICAL FIELD

The present invention relates to the field of organic synthesis and, more specifically, it concerns a process for preparing a compound of formula (I). Moreover, the present invention comprises also analytical methods of measuring the amount of thiol or thioether compounds having less than 6 carbon atoms in the thiol of formula R—SH and a composition of matter comprising at most 50 μg/kg of thiol or thioether compounds having less than 6 carbon atoms and at most 1 ppm of any compound of formula (I′).

### BACKGROUND OF THE INVENTION

The perfume industry has a particular interest for compositions or additives which are capable of prolonging or enhancing the perfuming effect for a certain period of time. It is particularly desirable to obtain long-lasting properties in order to enhance the consumer experience. Long-lasting perfumes are desirable for various applications, as for example fine or functional perfumery or cosmetic preparations. The washing and softening of textiles are particular fields in which there is a constant need to enable the effect of active substances, in particular perfumes, or perfuming compositions, to be effective for a certain period of time after washing, softening and drying. Indeed, many active substances which are particularly suitable for this type of application are known to lack tenacity on laundry, or do not remain on the laundry when rinsed, with the result that their perfuming effect is experienced only briefly and not very intensely. Given the importance of this type of application in the perfume industry, research in this field has been sustained, in particular with the aim of finding new, and more effective solutions to the aforementioned problems.

Delivery systems have been developed with this regard, such as precursor compounds, also called profragrances or properfumes, which release active material by a chemical reaction during or after application (using O₂, light, enzymes, water (pH) or temperature as the release trigger). Towards this goal, β-thio carbonyl profragrance derivatives of formula (I) herein below described, reported in WO 03/049666, were developed and in particular 3-(dodecylthio)-1-[(1RS,2SR)-2,6,6-trimethyl-3-cyclohexen-1-yl]-1-butanone also known as Haloscent® D (origin: Firmenich SA).

However, the olfactive quality of compound of formula (I) may be significantly affected by traces of impurities undetected by general analytic methods compromising its use in consumer products. In the meantime, compounds of formula (I) can decompose under general purification methods making further purification steps counterproductive.

So there is a need to develop an approach toward compounds of formula (I) while limiting the generation of off-notes and also to develop analytical method allowing to detect impurities present in the starting material and responsible for off-notes in the final product.

The present invention provides a solution to the above problem by preparing compounds of formula (I) by reacting compounds of formula (III) with a thiol of formula R—SH comprising at most 50 μg/kg of thiol or thioether compounds having less than 6 carbon atoms. In addition an analytical method has been developed in order to detect even tiny amount of thiol or thioether compounds having less than 6 carbon atoms.

### SUMMARY OF THE INVENTION

The invention relates to a novel process allowing the preparation of compounds of formula (I), through the 1,4-addition (Michael-type addition) of a thiol of formula R—SH on an enal, enone or alpha, beta-unsaturated carboxylic ester of formula (III), without the presence of off-notes while limiting the purification steps leading to the decomposition of compounds of formula (I), and so, leading to a decrease in yield.

So, the first object of the present invention is a method of measuring the amount of thiol or thioether compounds having less than 6 carbon atoms in the thiol of formula R—SH, wherein R represents a linear or branched alkyl group having from 8 to 15 carbon atoms, optionally comprising a carboxylic acid or a C1-8 carboxylic ester functional group, which is not directly linked to the sulfur atom, by Gas-chromatography-Mass Spectrometry (GC-MS) combined with solid phase microextraction (SPME) wherein the SPME is performed with the sample being cooled down to a temperature comprised between −100° C. to −50° C.

A second object of the present invention is a process for the preparation of a compound of formula

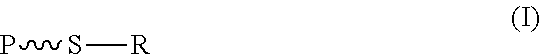

wherein the wavy line indicates the location of the bond between said P and the sulfur atom;
P represents a radical susceptible of generating an odoriferous α,β-unsaturated ketone, aldehyde or carboxylic ester and is represented by the formula

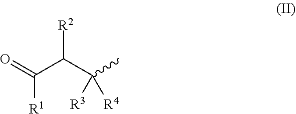

in which the wavy line indicates the location of the bond between said P and S;
R¹ represents a hydrogen atom, a C₁ to C₆ alkoxyl radical or a C₁ to C₁₅ linear, cyclic or branched alkyl, alkenyl or alkadienyl radical, optionally substituted by one to four C₁ to C₄ alkyl groups; and
R², R³ and R⁴ represent independently of each other a hydrogen atom, an aromatic ring, or a C₁ to C₁₅ linear, cyclic or branched alkyl, alkenyl or alkadienyl radical, possibly substituted by C₁ to C₄ alkyl groups; or two, or three, of the groups R¹ to R⁴ are bonded together to form a saturated or unsaturated ring having 5 to 20 carbon atoms and including the carbon atom to which said R¹, R², R³ or R⁴ groups are bonded, this ring being possibly substituted by C₁ to C₈ linear, branched or cyclic alkyl or alkenyl groups;
R represents a linear or branched alkyl group having from 8 to 15 carbon atoms, optionally comprising a carboxylic acid or a C₁-8 carboxylic ester functional group which is not directly linked to the sulfur atom;
   comprising the step of reacting a compound of formula

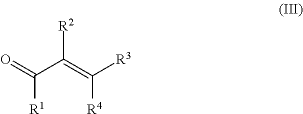

wherein R¹, R², R³ and R⁴ have the same meaning as defined above with a thiol of formula R—SH wherein R has the same meaning as defined above
characterized in that the thiol comprises at most 50 μg/kg of thiol or thioether compounds having less than 6 carbon atoms.

A third object of the present invention is a composition of matter comprising a compound of formula (I), at most 50 μg/kg of thiol or thioether compounds having less than 6 carbon atoms, and at most 1 ppm of any compound of formula

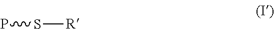

wherein the wavy line indicates the location of the bond between said P and the sulfur atom; P has the same meaning as defined above and R′ represents an alkyl group having from 1 to 5 carbon atoms.

### DESCRIPTION OF THE INVENTION

The invention relates to a novel process for the preparation of compounds of formula (I) devoid of any off-note while maintaining a high yield. The invention process represents an efficient route toward a compound of formula (I) without the presence of impurities badly impacting the organoleptic properties of this compound. Indeed, it has been surprisingly discovered that the presence of thiol or thioether compounds having less than 6 carbon atoms in a thiol of formula R—SH used to prepare compound of formula (I) is responsible for undesired off-notes in the final compound of formula (I) which could not be removed without decreasing the product yield.

So, a first object of the present invention is a process for the preparation of a compound of formula

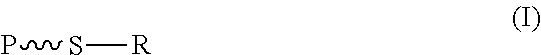

wherein the wavy line indicates the location of the bond between said P and the sulfur atom;
P represents a radical susceptible of generating an odoriferous α,β-unsaturated ketone, aldehyde or carboxylic ester and is represented by the formula

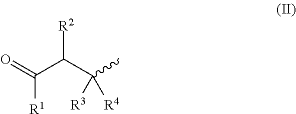

in which the wavy line indicates the location of the bond between said P and S; R¹ represents a hydrogen atom, a C₁ to C₆ alkoxyl radical or a C₁ to C₁₅ linear, cyclic or branched alkyl, alkenyl or alkadienyl radical, optionally substituted by one to four C₁ to C₄ alkyl groups; and
R², R³ and R⁴ represent independently of each other a hydrogen atom, an aromatic ring, or a C₁ to C₁₅ linear, cyclic or branched alkyl, alkenyl or alkadienyl radical, possibly substituted by C₁ to C₄ alkyl groups; or two, or three, of the groups R¹ to R⁴ are bonded together to form a saturated or unsaturated ring having 5 to 20 carbon atoms and including the carbon atom to which said R¹, R², R³ or R⁴ groups are bonded, this ring being possibly substituted by C₁ to C₈ linear, branched or cyclic alkyl or alkenyl groups;
R represents a linear or branched alkyl group having from 8 to 15 carbon atoms, optionally comprising a carboxylic acid or a C₁₋₈ carboxylic ester functional group which is not directly linked to the sulfur atom;
   comprising the step of reacting a compound of formula

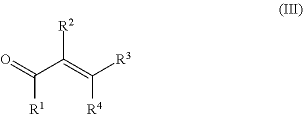

wherein R¹, R², R³ and R⁴ have the same meaning as defined above with a thiol of formula R—SH wherein R has the same meaning as defined above characterized in that the thiol comprises at most 50 μg/kg of thiol or thioether compounds having less than 6 carbon atoms.

For the sake of clarity, by the expression “thiol or thioether compounds having less than 6 carbon atoms”, or the similar, it is meant the normal meaning understood by a person skilled in the art, i.e. an organosulfur compound comprising a —SH functional group and having less than 6 carbon atoms or an organosulfur compound comprising a —S— functional group and having less than 6 carbon atoms.

According to any embodiment of the invention, the invention's process comprises the step of measuring the amount of thiol or thioether compounds having less than 6 carbon atoms in the thiol of formula R—SH by Gas-chromatography-Mass Spectrometry (GC-MS) combined with solid phase microextraction (SPME) wherein the SPME is performed with the neat sample being cooled down to a temperature comprised between −100° C. to −50° C. The SPME fiber is preferably a 50/30 μm divinylbenzene/Carboxen on polydimethylsiloxane on a StableFlex fiber (part No 57329-U, Supelco) or 50/30 μm divinylbenzene/Carboxen on polydimethylsiloxane on a 2 cm StableFlex fiber. Alternative fibers that may be used are: 65 μm polydimethylsiloxane/divinylbenzene or 75 μm/85 μm Carboxen/polydimethylsiloxane.

According to any embodiment of the invention, the thiol or thioether compounds having less than 6 carbon atoms may be selected from the group consisting of 1-propanethiol, 2-propanethiol, 2-methyl-1-propanethiol, 2-methyl-2-propanethiol, 1-butanethiol, tetrahydrothiophene and a mixture thereof.

According to any embodiment of the invention, the thiol of formula R—SH comprises
a) at most 5 μg/kg of 1-propanethiol
b) at most 5 μg/kg of 2-propanethiol
c) at most 5 μg/kg of 2-methyl-1-propanethiol
d) at most 20 μg/kg of 2-methyl-2-propanethiol
e) at most 5 μg/kg of 1-butanethiol; and
f) at most 10 μg/kg of tetrahydrothiophene.

In a particular embodiment of the invention, the thiol of formula R—SH comprises
a) at most 2.5 μg/kg of 1-propanethiol
b) at most 3.5 μg/kg of 2-propanethiol
c) at most 2.5 μg/kg of 2-methyl-1-propanethiol
d) at most 18 μg/kg of 2-methyl-2-propanethiol
e) at most 2.5 μg/kg of 1-butanethiol; and
f) at most 5 μg/kg of tetrahydrothiophene.

In a particular embodiment of the invention, the thiol of formula R—SH comprises
a) at most 1 μg/kg of 1-propanethiol
b) at most 3 μg/kg of 2-propanethiol
c) at most 1 μg/kg of 2-methyl-1-propanethiol
d) at most 18 μg/kg of 2-methyl-2-propanethiol
e) at most 1 μg/kg of 1-butanethiol; and
f) at most 3 μg/kg of tetrahydrothiophene.

In a particular embodiment, the thiol of formula R—SH is devoid of traces of 1-propanethiol, 2-propanethiol, 2-methyl-1-propanethiol, 2-methyl-2-propanethiol, 1-butanethiol or tetrahydrothiophene.

According to any embodiment of the invention, the thiol of formula R—SH may be purified by distillation before reacting with the compound of formula (III). In particular, the distillation may be a batch or continuous distillation, in particular, a batch distillation. The distillation may be performed by removing the low boiling point fractions (topping), by selecting the desired fractions (fractional distillation) or by flash distillation using falling (wiped) film evaporators.

According to any embodiment of the invention, R may be a linear alkyl group having from 8 to 15 carbon atoms. In particular R may be a linear alkyl group having from 10 to 14 carbon atoms, and in particular a n-dodecyl group; i.e. R—SH may be 1-dodecanethiol.

As “odoriferous α,β-unsaturated ketone, aldehyde or carboxylic ester”, the expression used in the definition of P, it is understood an α,β-unsaturated ketone, aldehyde or carboxylic ester, which is recognized by a skilled person as being used in perfumery as perfuming ingredient. In general, said odoriferous α,β-unsaturated ketone, aldehyde or carboxylic ester is a compound having from 8 to 20 carbon atoms, or even more preferably between 10 and 15 carbon atoms.

Similarly, it is not possible to provide an exhaustive list of the currently known odoriferous compounds of formula (III), which can be used in the synthesis of the compound of formula (I). However, the following can be named as preferred examples: alpha-damascone, beta-damascone, gamma-damascone, delta-damascone, alpha-ionone, beta-ionone, gamma-ionone, delta-ionone, beta-damascenone, 1-[6-ethyl-2,6-dimethyl-3-cyclohexen-1-yl]-2-buten-1-one, 3-methyl-5-propyl-2-cyclohexen-1-one, 2-methyl-5-(1-propen-2-yl)-2-cyclohexen-1-one, 2,5-dimethyl-5-phenyl-1-hexen-3-one, 1-(5,5-dimethyl-1-cyclohexen-1-yl)-4-penten-1-one, 3,7-dimethylocta-2,6-dienal, 8-methyl-alpha-ionone or 10-methyl-alpha-ionone, 2-octenal, 1-(2,2,3,6-tetramethylcyclohexyl)but-2-en-1-one, 4-(2,2,3,6-tetramethylcyclohexyl)but-3-en-2-one, 2-cyclopentadecen-1-one, 4,4a-dimethyl-6-(1-propen-2-yl)-4,4a,5,6,7,8-hexahydro-2(3H)-naphthalenone, (E)-3-phenylprop-2-enal, 2,6,6-trimethylspiro[bicyclo[3.1.1]heptane-3,1′-cyclohexan]-2′-en-4′-one, ethyl 2,4-decadienoate, ethyl 2-octenoate, methyl 2-nonenoate, ethyl 2,4-undecadienoate, 4-methylpent-3-en-2-one, oct-2-en-4-one, and methyl 5,9-dimethyl-2,4,8-decatrienoate.

According to any embodiment of the invention, the compound of formula (I) may be in a form of any one of its stereoisomers or a mixture thereof and the compound of formula (III) may be in a form of any one of its stereoisomers or a mixture thereof. For the sake of clarity, by the expression “any one of its stereoisomers or a mixture thereof”, or the similar, it is meant the normal meaning understood by a person skilled in the art, i.e. that the compound of formula (I) and (III) can be a pure enantiomer or diastereomer. In other words, the compound of formula (I) and (III) may possess several stereocenters and each of said stereocenter can have two different configurations (e.g. R or S). The compound of formula (I) and (III) may even be in the form of a pure enantiomer or in the form of a mixture of enantiomers or diastereoisomers. The compound of formula (I) and (III) can be in a racemic form or scalemic form. Therefore, the compound of formula (I) and (III) can be one stereoisomers or in the form of a composition of matter comprising, or consisting of, various stereoisomers.

According to any one of the above embodiments of the invention, the compound of formula (III) can be in the form of its E or Z isomer or of a mixture thereof, e.g. the invention comprises compositions of matter consisting of one or more compounds of formula (III), having the same chemical structure but differing by the configuration of the double bond.

According to any embodiment of the invention, P is a radical selected from the group consisting of formulae (P-1) to (P-14), in the form of any one of their isomers:

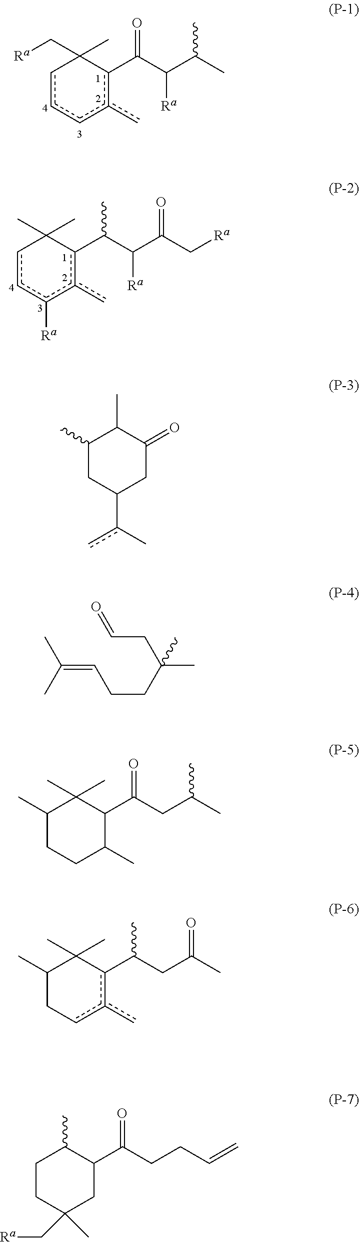

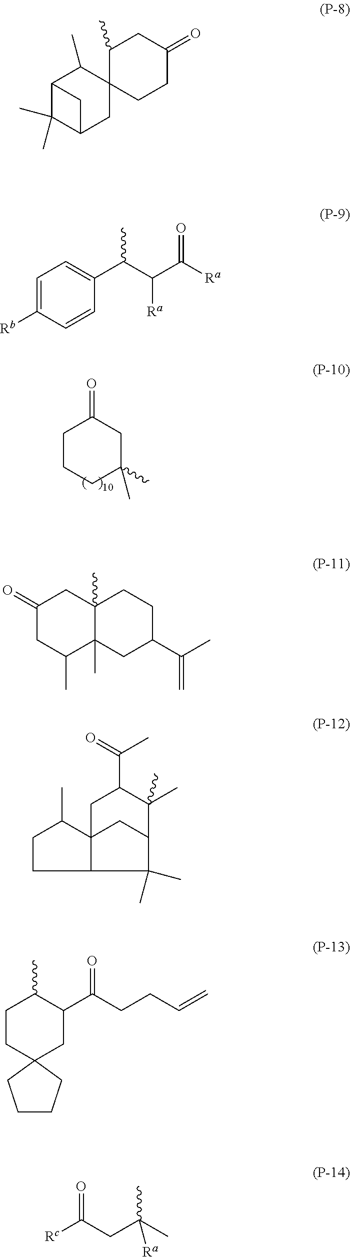

in which formulae the wavy lines have the meaning indicated above and the dotted lines represent a single or double bond, R^{a} being a hydrogen atom or a methyl group and R^{b} representing a hydrogen atom, a hydroxyl or methoxy group or a C₁-C₄ linear or branched alkyl group and R^{C} representing a hydrogen atom or a C₁-C₄ linear or branched alkyl group. Accordingly, the compound of formula (I) wherein P is selected from the group consisting of formulae (P-1) to (P-14) is prepared by reacting compound of formula

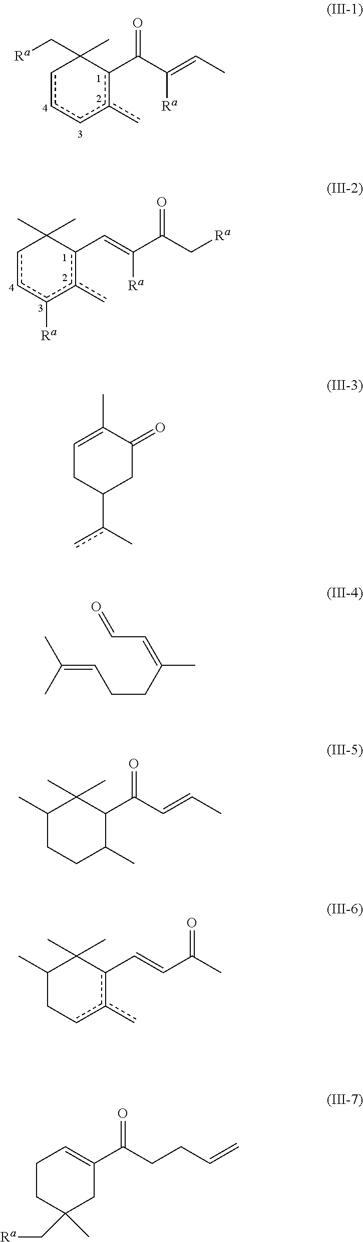

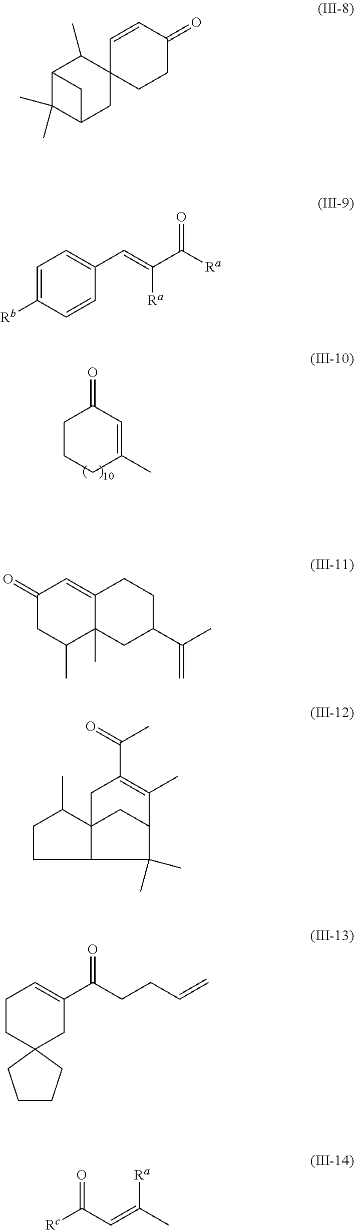

wherein the dotted lines, R^{a}, R^{b} and R^{c} have the same meaning as defined above.

For the sake of clarity, by the expression “wherein one dotted line represents a single or double bond”, or the similar, it is meant the normal meaning understood by a person skilled in the art, i.e. that the whole bonding (solid and dotted line) between the carbon atoms connected by said dotted line is a carbon-carbon single bond or a carbon-carbon double bond.

In a particular embodiment, P represents a radical selected from the group consisting of formulae

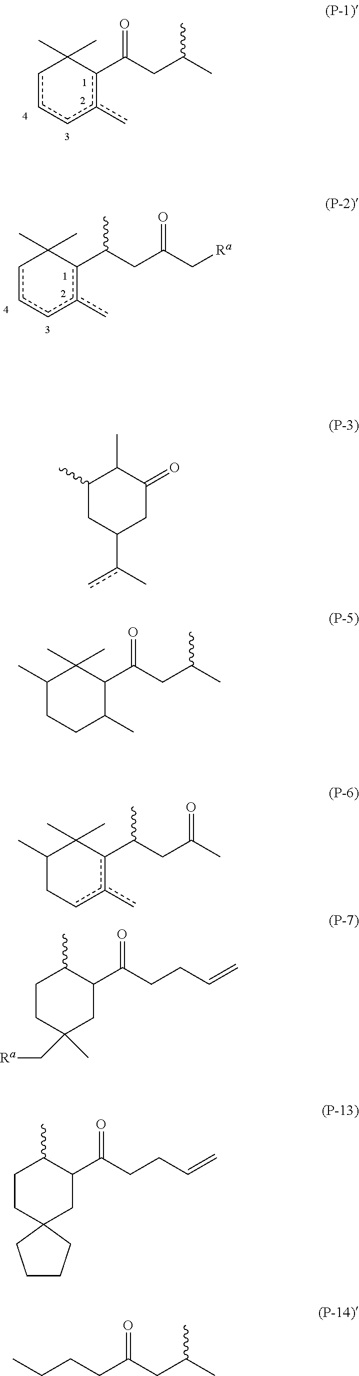

wherein the wavy lines have the meaning indicated above and the dotted lines represent a single or double bond, and R^{a} being a hydrogen atom or a methyl group. Accordingly, the compound of formula (I) wherein P is selected from the group consisting of formulae (P-1)′, (P-2)′, (P-3), (P-5), (P-6), (P-7), (P-13) and (P-14)′ is prepared by reacting compound of formula

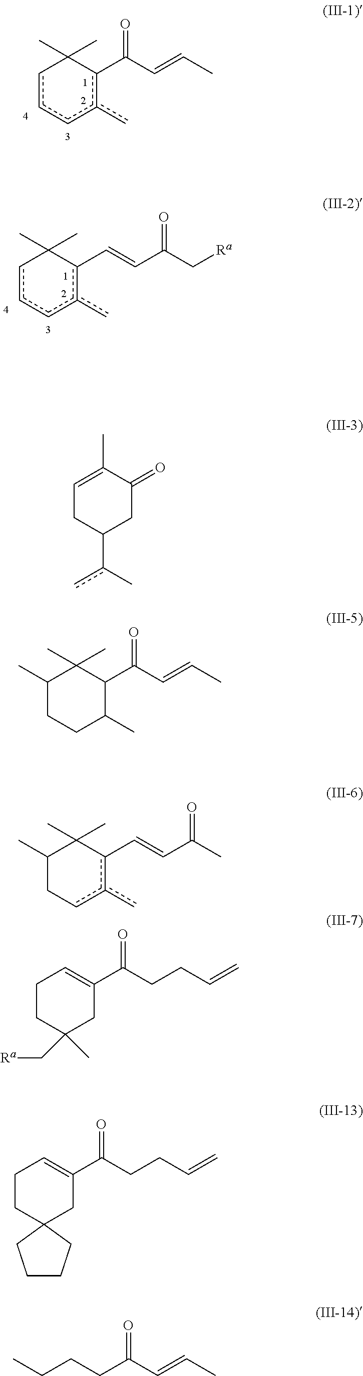

wherein the dotted lines and R^{a} have the same meaning. as defined above.

In a particular embodiment, P represents a radical selected from the group consisting of formulae (P-1), (P-2), (P-1)′, (P-2)′, (P-3), (P-7), (P-13), (P-14) or (P-14)′ as defined above. In particular, P may be (P-1), (P-2), (P-3), (P-7), (P-13) or (P-14). Preferably, P represents a radical selected from the group consisting of formulae (P-1), (P-1)′, (P-2), (P-2)′, (P-3) or (P-14)′ as defined above.

In a particular embodiment, the compound of formula (I) is a compound selected from the group consisting of formulae a) to d)

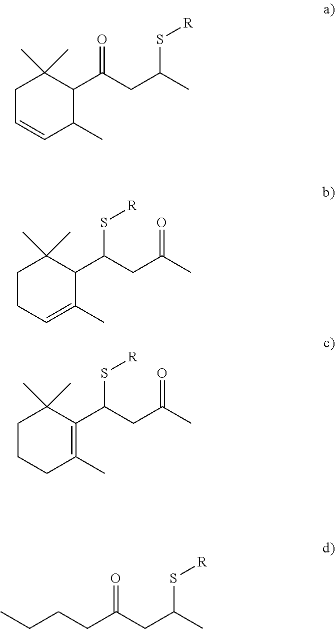

wherein R has the same menaing as defined above.

The compound of formula a) is obtained from the reaction of a compound of formula (III), wherein the compound of formula (III) is delta-damascone. Said compound of formula a) may preferably be (±)-trans-3-(dodecylthio)-1-(2,6,6-trimethyl-3-cyclohexen-1-yl)-1-butanone. Delta-damascone is also known as 1-[(1RS,2SR)-2,6,6-trimethyl-3-cyclohexen-1-yl]-2-buten-1-one.

The compound of formula b) or c) is obtained from the reaction of a compound of formula (III), wherein the compound of formula (III) is ionone. Said compound of formula b) or c) may be present as an isomeric mixture of formula b) and formula c). The isomeric mixture may have a weight ratio of formula b) and formula c) from 40:60 to 60:40. In particular, the isomeric mixture may have a weight ratio of formula b) and formula c) of about 55:45. In particular, said compound of formula b) or c) releases two isomers of ionone as fragrance compound.

In particular, the compound of formula b) is obtained from the reaction of a compound of formula (III), wherein the compound of formula (III) is alpha-ionone. Said compound of formula b) may preferably be (±)-4-(dodecylthio)-4-(2,6,6-trimethyl-2-cyclohexen-1-yl)-2-butanone. Alpha-ionone is also known as (±)-(3E)-4-(2,6,6-trimethyl-2-cyclohexen-1-yl)-3-buten-2-one.

In particular, the compound of formula c) is obtained from the reaction of a compound of formula (III), wherein the compound of formula (III) is beta-ionone. Said compound of formula c) may preferably be (±)-4-(dodecylthio)-4-(2,6,6-trimethyl-1-cyclohexen-1-yl)-2-butanone. Beta-ionone is also known as (3E)-4-(2,6,6-trimethyl-1-cyclohexen-1-yl)-3-buten-2-one.

The compound of formula d) is obtained from the reaction of a compound of formula (III), wherein the compound of formula (III) is oct-2-en-4-one. Said compound of formula d) may preferably be (±)-2-(dodecylthio)octan-4-one. Oct-2-en-4-one may be used as its (E)- or (Z)-isomers, or as mixtures thereof, with the (E)-isomer being preferred.

According to any embodiment of the invention, and as non-limiting examples of compounds of formula (I) one may cite the following: 3-(dodecylthio)-1-(2,6,6-trimethylcyclohex-3-en-1-yl)butan-1-one, 2-(dodecylthio)-4-octanone, 3-(dodecylthio)-1-(2,6,6-trimethylcyclohex-2-en-1-yl)butan-1-one, 4-(dodecylthio)-4-(2,6,6-trimethylcyclohex-2-en-1-yl)butan-2-one and 4-(dodecylthio)-4-(2,6,6-trimethylcyclohex-1-en-1-yl)butan-2-one and 4-(dodecylthio)-4-methylpentan-2-one.

According to any embodiment of the invention, the reaction between the compound of formula (III) and the thiol of formula R—SH may be conducted in the presence of any reagent commonly used for 1,4-additions such as a base, Lewis acid, transitional metal or organocatalyst. Particularly, said reaction may be performed in presence of a base. Particularly, the base is a tertiary amine. Non-limiting examples of suitable bases may include triethylamine, trimethylamine, urotropine, N,N-diisopropylethylamine, 1,8-diazobicyclo(5.4.0)undec-7-ene, or a mixture thereof.

The base can be added into the reaction medium of the invention's process in a large range of concentrations to form a compound of formula (I). As non-limiting examples, one can cite, as base concentration values those ranging from 0.001 to 2 equivalents, relative to the total amount of compound of formula (III). Particularly, the base concentration may be comprised between 0.005 to 1.5 equivalents relative to the total amount of compound of formula (III). It goes without saying that the process works also with more base. However the optimum concentration of base will depend, as the person skilled in the art knows, on the nature of the latter, on the nature of compound of formula (III) and of thiol of formula R—SH, on the temperature and on the desired time of reaction.

The thiol of formula R—SH can be added into the reaction medium of the invention's process in a large range of concentrations to form compound of formula (I). As non-limiting examples, one can cite, as thiol concentration values those ranging from 0.5 to 5 equivalents, relative to the total amount of compound of formula (III). Particularly, the thiol concentration may be comprised between 0.75 to 3 equivalents relative to the total amount of compound of formula (III). It goes without saying that the process works also with more thiol of formula R—SH. However the optimum concentration of thiol of formula R—SH will depend, as the person skilled in the art knows, on the nature of the latter, on the nature of compound of formula (III) and of base, on the temperature and on the desired time of reaction.

According to any one of the invention's embodiments, the invention's process to form a compound of formula (I) is carried out at a temperature comprised between −10° C. and 150° C. In particular, the temperature is in the range between 30° C. and 70° C. Of course, a person skilled in the art is also able to select the preferred temperature as a function of the melting and boiling point of the starting and final products as well as the desired time of reaction or conversion.

The invention's process to form a compound of formula (I) can be carried out in the presence or absence of a solvent. When a solvent is required or used for practical reasons, then any solvent current in such reaction type can be used for the purposes of the invention. Non-limiting examples include C₆₋₁₂ aromatic solvents such as xylene, toluene, 1,3-diisopropylbenzene, cumene or pseudocumene, or mixtures thereof, hydrocarbon solvents such as cyclohexane, heptane or mixtures thereof, nitrile solvents such as acetonitrile, esteral solvents such as ethyl acetate or ethereal solvents such as tetrahydrofuran, diethyether, methyl tetrahydrofuran or mixtures thereof. The choice of the solvent is a function of the nature of the substrates and/or base and the person skilled in the art is well able to select the solvent most suitable in each case to optimize the reaction. Particularly, the invention's process to form a compound of formula (I) is carried out in absence of solvent.

The invention's process to form a compound of formula (I) is carried out under batch, semi-batch or continuous conditions.

The invention's process to form a compound of formula (I) may further comprise the step of purifying the compound of formula (I). The person skilled in the art is well aware of the most adapted purification method. Non-limiting examples of suitable purification methods may include washing-drying, vacuum distillation in particular vacuum distillation using wiped-film distillation or chromatography.

The presence of a tiny amount of sulfur-containing hydrocarbons impurities present in the compound of formula (I), which are responsible for undesirable off-notes in the final product, has to be minimized. It has been surprisingly discovered that there is a link between the presence of thiol or thioether compounds having less than 6 carbon atoms in the thiol of formula R—SH and the presence of off-notes in the final product, which could surprisingly not be removed by purification at the end of the process. The detection of thiol or thioether compounds having less than 6 carbon atoms in the thiol of formula R—SH cannot be performed using a simple GC method since it is not be able to detect these very powerful odorants with the relevant sensitivity. A novel analytical method has been developed for this specific purpose.

So another object of the invention is a method of measuring the amount of thiol or thioether compounds having less than 6 carbon atoms in the thiol of formula R—SH, wherein R has the same meaning as defined above, by Gas-chromatography-Mass Spectrometry (GC-MS) combined with solid phase microextraction (SPME) wherein the SPME is performed with the neat sample cooled down to a temperature comprised between −100° C. to −50° C. The SPME fiber is a 50/30 μm DVB/CAR/PDMS Stableflex, (part No 57329-U, Supelco).

According to any embodiment of the invention, the thiol or thioether compounds having less than 6 carbon atoms detected by this method are selected from the group consisting of 1-propanethiol, 2-propanethiol, 2-methyl-1-propanethiol, 2-methyl-2-propanethiol, 1-butanethiol and tetrahydrothiophene.

According to any embodiment of the invention, the MS of the GC/MS is a triple quadrupole, a quadrupole Time-of-Flight (Q-TOF) or a quadrupole orbitrap. The gas used is helium.

According to any embodiment of the invention, the mode of acquiring GC-MS data is Selected Ion Monitoring (SIM) or Selected Reaction Monitoring (SRM).

According to a particular embodiment, the GC chromatography uses a non-polar GC column (DB-1, SPB-1) or possibly a GC column of moderate polarity (DB-5). The GC gradient oven has to start at low temperature so as to separate the volatile sulfur compounds adequately.

A further object of the invention is a composition of matter comprising a compound of formula (I), at most 50 μg/kg of thiol or thioether compounds having less than 6 carbon atoms and at most 1 ppm of any compound of formula

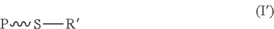

wherein the wavy line indicates the location of the bond between said P and the sulfur atom; P has the same meaning as defined above R′ represents an alkyl group having from 1 to 5 carbon atoms.

The analytical method used to measure the amount of any product of formula (I′) in the compound of formula (I) is HPLC/MS or UPLC/MS. The product of formula (I) is typically injected at a concentration of up to 10% onto a C₁₈ column eluted with water/acetonitrile. The flow to the MS is typically diverted when the major product (I) elutes which is always at retention times higher than those of smaller products of formula (I′). The MS may be selected from the group consisting of triple quadrupole, Q-Tof and Q-Orbitrap. When the signal corresponding to the molecular ion of (I′) is not seen, the corresponding product is generally below 10 μg/kg.

### EXAMPLES

The invention will now be described in further detail by way of the following examples, wherein the abbreviations have the usual meaning in the art, the temperatures are indicated in degrees centigrade (° C.). The solvents were dried by conventional procedures and distilled under an argon atmosphere. NMR spectra were recorded at 20° C. on Bruker AV 300, AV 400, or AV 500 MHz spectrometers. Chemical shifts are reported in ppm relative to solvent signals (chloroform, δ_{H}=7.26 ppm, δ_{C}=77.0 ppm). The signal assignment was ensured by recording ¹H, ¹H-COSY, -NOESY, ¹³C, ¹H-HSQC and -HMBC experiments.

### Example 1

### Measurement of the Amount of Thiol or Thioether Having Less Than 6 Carbon Atoms in Dodecanethiol

3 batches of dodecanethiol have been analyzed by solid-phase microextraction (SPME) and GC/MS as described below. Batch 3 was obtained by distillation of batch 2 as reported below.

### 30 a) Typical Dodecanethiol Distillation Procedure

600 g of dodecanethiol were charged into a round bottom flask equipped with a thermowell, magnetic stir bar and connected to a fractionation column The fractionation column was filled with a structured packing with an efficiency of approx. 15 theoretical plates. The distillation commenced under reduced pressure (2-3 mmHg) and the pot was heated until a maximum temperature of 161° C. The following fractions were obtained.

| T(pot) | T(vap) | Pressure | wt | |
|---|---|---|---|---|
| (° C.) | (° C.) | (mmHg) | (g) | |
| 134 | 112 | 2.3 | 134.8 | front fraction |
| 135 | 114 | 2.2 | 325 | heart fraction |
| 161 | 87 | 2.2 | 95.6 | heart fraction |
| 161 | — | 2.2 | 38 | heavy fraction |

### b) SPME Method

SPME was performed using a grey fiber (50/30μm DVB/CAR/PDMS Stableflex, part No 57329-U, Supelco). Manual SPME extractions at −80° C. (15 min) were performed by manual swirling a 20 mL headspace vial containing dodecanethiol (1 g) in a dry ice/ethanol bath until dodecanethiol solidified on the glass wall of the vial. Then the fiber was exposed to the headspace of the sample for 15 min while the vial was still partially immerged in the dry ice/ethanol bath. This SPME method assured the highest sensitivity.

Alternatively, SPME can be performed at RT using an automatic sampler (combi-PAL, CTC) but with a lower sensitivity.

### c) GC/MS Method

A 7890/7000B triple quadrupole GC/MS system (Agilent) was used. The column was a SPB-1, 30 m, 0.25 mm ID, 0.25 μm film thickness (Supelco) eluted with He as a carrier gas at 0.7 mL/min, Oven: 40° C., 5 min then 5° C./min to 60° C. and finally 20° C./min to 250° C., 250° C. for 1.5 min. The parent/product ions and collision energy (expressed in eV) for the different products were as followed:

1-butanethiol: 90/56 (3 eV) and 90/41 (6 eV); 1-propanethiol: 76/60 (3 eV) and 76/47 (6 eV); 2-methyl-1-propanethiol: 90/56 (4 eV) and 90/41 (6 eV); 2-propanethiol: 76/60 (2 eV) and 76/43 (6 eV); 2-methyl-2-propanethiol: 90/57 (2 eV) and 90/41 (2 eV); tetrahydrothiophene: 88/60 (14 eV) and 88/54 (9 eV)

The SPME protruding fiber was exposed for 5 min before GC/MS injection using the autosampler PAL automatized procedure. Splitless injection was used.

### d) Results

TABLE 1
Measurement of thiol or thioether compounds having
less than 6 carbon atoms in dodecanthiol

| | | | Batch 3¹⁾ | Batch 3¹⁾ | Batch 3¹⁾ |
|---|---|---|---|---|---|
| Thiols content | | | front | heart | heavy |
| expressed in μg/kg | Batch 1 | Batch 2 | fraction | fraction | fraction |
| 1-butanethiol | 0 | 19 | 129 | 34 | 0 |
| 1-propanethiol | 0 | 153 | 45 | 2 | 0 |
| 2-methyl-1-propanethiol | 0 | 4 | 1 | 1 | 1 |
| 2-propanethiol | 3 | 142 | 26 | 3 | 2 |
| 2-methyl-2-propanethiol | 18 | 523 | 142 | 13 | 3 |
| Tetrahydrothiophene | 3 | 4834 | 4508 | 183 | 2 |

¹⁾Batch 3 obtained by distillation of batch 2. front, heart and heavy fractions

Batch 1 and the heavy fractions of Batch 3 represent dodecanethiol batches to be used in the invention process. Batch 2 and the front and heart fractions of Batch 3 represent dodecanethiol batches to be used to prepare comparative examples.

### Example 2

### Preparation of a Compound of Formula (I) and Analysis

### a) Preparation of Compound of Formula (I)

A solution of δ-damascone (10.0 g; 52.1 mmol) and 1-dodecanethiol using 5 different batches as reported below (8.42 g, 41.7 mmol) in THF (150 ml) was treated with DBU (7.92 g; 52.1 mmol) and stirred at 45° C. for 90 min The reaction solution was treated with 5% aqueous HCl, extracted twice with ether, washed with H₂O, saturated aqueous NaHCO₃, then brine, dried over Na₂SO₄, and concentrated at 70° C./0.01 mbar. Yield of crude product: 16.2 g (99%).

¹H-NMR: 0.84-0.92 (m, 6 H); 0.93-1.02 (4 s, 6 H); 1.26 (m, 16 H); 1.29 (m, 3 H); 1.36 (m, 2 H); 1.58 (m, 2 H); 1.69 (m, 1H); 1.96 (2 b, 1 H); 2.22 (m, 1 H); 2.50 (m, 3.5 H); 2.70 (m, 1 H); 2.90 (m, 0.5 H); 3.30 (m, 1 H); 5.43 (m, 1 H); 5.53 (m, 1 H).

¹³C-NMR: 212.4/5 (s); 131.8/9 (d); 124.1/2 (d); 62.9/63.0 (d); 55.2/3 (t); 41.7 (t); 34.1 (d); 33.0/2 (s); 31.9 (t); 31.6/8 (d); 30.9 (t); 29.8 (q); 29.0-29.8 (several t); 22.7 (t); 21.6/8 (q); 20.7 (q); 19.9 (q); 14.1 (q).

### b) Measurement of the Amount of Thiol or Thioether Having Less Than 6 Carbon Atoms in the Products of Formula (I)

The measurement of the amount of thiols having less than 6 carbon atoms in the products of formula (I) has been performed using the SPME GC/MS method described in example 1. The SPME sampling of (I) was performed at room temperature. The results are shown in Table 2.

All batches of (I) obtained with different dodecanethiol batches have also been analyzed by UPLC/MS to assess their content in compounds of formula (I′). The system was composed of an Acquity I-Class UPLC system (Waters) coupled to a QExactive Plus mass spectrometer (Thermo). The column was an Acquity BEH C18 (1.7 μm, 2.1×100 mm) eluted with 0.4 ml/min of H₂O/ACN 0.1% formic acid. The gradient was as follows: 0% B, 0.5 min ; 0-100% B in 10 min; 100% B, 1 min; equilibration for 1 min. The MS was operated in the negative ESI mode, voltage 3500V, probe heater (source) 300° C., capillary 300° C., sheath gas 48, aux gas 11. Full scan ddMS2 mode [150-2000 Da] was used for qualitative and quantitative analyses with stepped NCEs of 10, 30 and 45V. PRM by using an inclusion list and isolation width of 1.0 Da (no offset). The results are reported in Table 2.

TABLE 2
Measurement of thiol or thioether compounds having less than
6 carbon atoms and compounds of formula (I′) in 3-(dodecylthio)-
1-[2,6,6-trimethyl-3-cyclohexen-1-yl]-1-butanone

| Thiols content expressed in | | | |
|---|---|---|---|
| μg/kg (SPME GC/MS | | | Batch 3 |
| method) | Batch 1 | Batch 2 | front fraction |
| 1-butanethiol | <1 | <1 | <1 |
| 1-propanethiol | <1 | <1 | <1 |
| 2-methyl-1-propanethiol | <1 | <1 | <1 |
| 2-propanethiol | <1 | <1 | <1 |
| 2-methyl-2-propanethiol | <1 | <1 | 1-4 |
| Tetrahydrothiophene | <1 | 390 | 1575 |
| Products of formula (I′) | No (I′) | Propyl and | Not analyzed |
| (UPLC/MS method) | detected | butyl (I′) | |
| | | detected | |

The presence of 1-propanethiol, 2-propanethiol, 2-methyl-1-propanethiol, 2-methyl-2-propanethiol, 1-butanethiol was not detected in the final product even though the compound of formula (I) prepared with those batches possess strong off-notes as shown below. Those results confirm that the analysis of small thiols has to be performed beforehand on the starting material dodecanethiol. Indeed, even undetected traces of those compound in the final product is detrimental.

### Example 3

### Organoleptic Evaluation of Compounds of Formula (I)

The batches obtained in Example 2 have been evaluated by three trained panelists by smelling above the freshly open bottle. The panelists were asked to rate the sulfur odor intensity on a scale from 1 to 10, 1 corresponding to odorless and 10 corresponding to a very strong odor.

The results are reported in Table 3.

TABLE 3
Organoleptic evaluation of three batches of 3-(dodecylthio)-1-[2,6,6-
trimethyl-3-cyclohexen-1-yl]-1-butanone prepared in example 2

| | | | Batch 3 | Batch 3 | Batch 3 |
|---|---|---|---|---|---|
| | | | front | Heart | Heavy |
| From dodecanthiol: | Batch 1 | Batch 2 | fraction | fraction | fraction |
| Sulfur odor | 3 | 7 | 9 | 5 | 3 |
| Intensity | | | | | |

Only compound of formula (I) prepared with batches 1 and heavy fraction of batch 3 of dodecanethiol imparted a very low undesired sulfur odor.

1. A method of measuring the amount of thiol or thioether compounds having less than 6 carbon atoms in the thiol of formula R—SH, wherein R represents a linear or branched alkyl group having from 8 to 15 carbon atoms, optionally comprising a carboxylic acid or a C₁₋₈ carboxylic ester functional group, which is not directly linked to the sulfur atom, by Gas-chromatography-Mass Spectrometry (GC-MS) combined with solid phase microextraction (SPME) wherein the SPME is performed with the sample cooled down to a temperature comprised between −100° C. to −50° C.

2. The method according to claim 1, wherein the thiol or thioether compounds having less than 6 carbon atom are selected from the group consisting of 1-propanethiol, 2-propanethiol, 2-methyl-1-propanethiol, 2-methyl-2-propanethiol, 1-butanethiol and tetrahydrothiophene.

3. The method according to claim 1, wherein the MS of the GC/MS is a triple quadrupole, a quadrupole Time-of-Flight (Q-TOF) or a quadrupole orbitrap.

4. The method according to claim 1, wherein the mode of acquiring GC-MS data is Selected Ion Monitoring (SIM) or Selected Reaction Monitoring (SRM).

5. A process for the preparation of a compound of formula 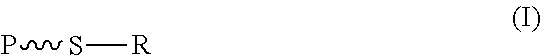
wherein the wavy line indicates the location of the bond between said P and the sulfur atom;
P represents a radical susceptible of generating an odoriferous α,β-unsaturated ketone, aldehyde or carboxylic ester and is represented by the formula 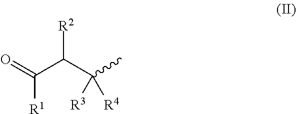
in which the wavy line indicates the location of the bond between said P and S;
R¹ represents a hydrogen atom, a C₁ to C₆ alkoxyl radical or a C₁ to C₁₅ linear, cyclic or branched alkyl, alkenyl or alkadienyl radical, optionally substituted by one to four C₁ to C₄ alkyl groups; and
R², R³ and R⁴ represent independently of each other a hydrogen atom, an aromatic ring, or a C₁ to C₁₅ linear, cyclic or branched alkyl, alkenyl or alkadienyl radical, possibly substituted by C₁ to C₄ alkyl groups; or two, or three, of the groups R¹ to R⁴ are bonded together to form a saturated or unsaturated ring having 5 to 20 carbon atoms and including the carbon atom to which said R¹, R², R³ or R⁴ groups are bonded, this ring being possibly substituted by C₁ to C₈ linear, branched or cyclic alkyl or alkenyl groups;
R represents a linear or branched alkyl group having from 8 to 15 carbon atoms, optionally comprising a carboxylic acid or a C₁₋₈ carboxylic ester functional group which is not directly linked to the sulfur atom;
comprising the step of reacting a compound of formula 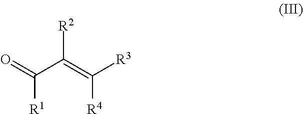
wherein R¹, R², R³ and R⁴ have the same meaning as defined above with a thiol of formula R—SH wherein R has the same meaning as defined above characterized in that the thiol comprises at most 50 μg/kg of thiol or thioether compounds having less than 6 carbon atoms.

6. The process according to claim 5, wherein the thiol of formula R—SH comprises
a) at most 5 μg/kg of 1-propanethiol
b) at most 5 μg/kg of 2-propanethiol
c) at most 5 μg/kg of 2-methyl-1-propanethiol
d) at most 20 μg/kg of 2-methyl-2-propanethiol
e) at most 5 μg/kg of 1-butanethiol; and
f) at most 10 μg/kg of tetrahydrothiophene.

7. The process according to claim 5, wherein the thiol of formula R—SH is devoid of traces of 1-propanethiol, 2-propanethiol, 2-methyl-1-propanethiol, 2-methyl-2-propanethiol, 1-butanethiol or tetrahydrothiophene.

8. The process according to claim 5, wherein the thiol of formula R—SH is purified by distillation before reacting with the compound of formula (III).

9. The process according to claim 5, wherein R is a linear alkyl group having from 10 to 14 carbon atoms.

10. The process according to claim 5, wherein the thiol of formula R—SH is 1-dodecanethiol.

11. The process according to claim 5, wherein P is a radical selected from the group consisting of formulae (P-1) to (P-14), in the form of any one of their isomers: 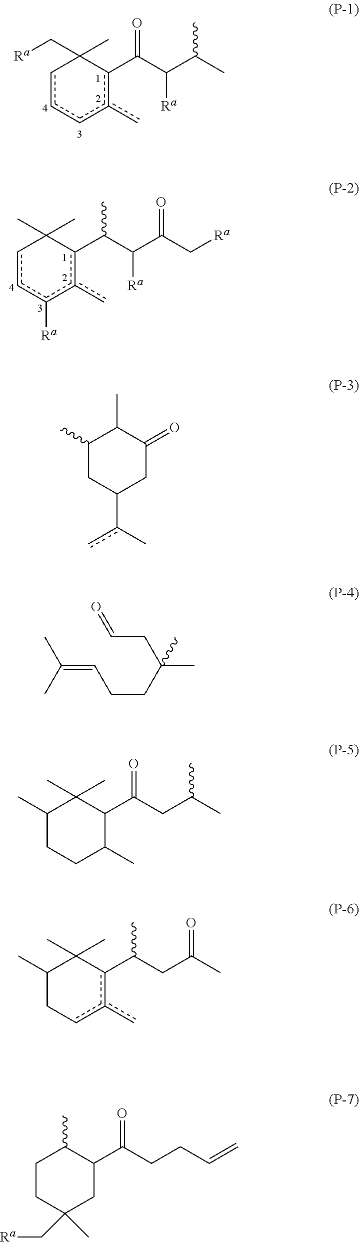 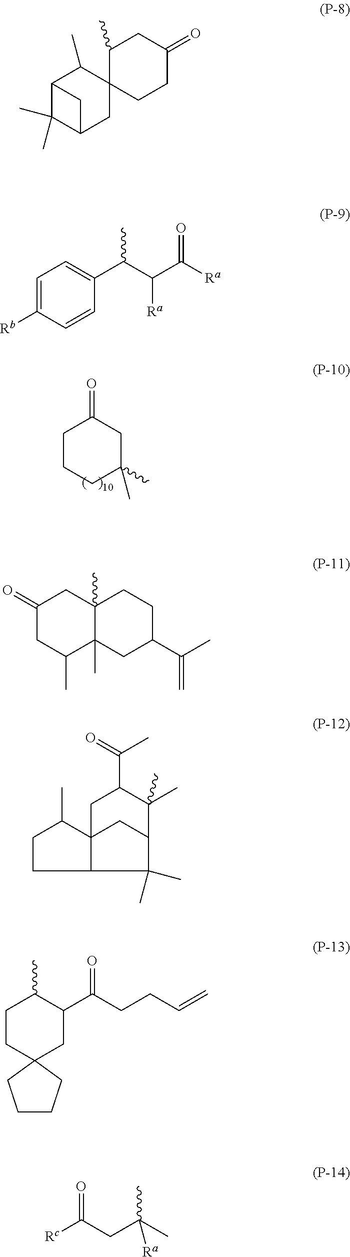
in which formulae the wavy lines have the meaning indicated above and the dotted lines represent a single or double bond, R^{a} being a hydrogen atom or a methyl group and R^{b} representing a hydrogen atom, a hydroxyl or methoxy group or a C₁-C₄ linear or branched alkyl group and R^{c} representing a hydrogen atom or a C₁-C₄ linear or branched alkyl group.

12. The process according to claim 11, wherein P is (P-1), (P-2), (P-3), (P-7), (P-13) or (P-14).

13. The process according to claim 5, wherein compound of formula (I) is selected from the group of 3-(dodecylthio)-1-(2,6,6-trimethylcyclohex-3-en-1-yl)butan-1-one, 2-(dodecylthio)-4-octanone, 3-(dodecylthio)-1-(2,6,6-trimethylcyclohex-2-en-1-yl)butan-1-one, 4-(dodecylthio)-4-(2,6,6-trimethylcyclohex-2-en-1-yl)butan-2-one and 4-(dodecylthio)-4-(2,6,6-trimethylcyclohex-1-en-1-yl)butan-2-one and 4-(dodecylthio)-4-methylpentan-2-one.

14. The process according to claim 5, wherein the reaction between compound of formula (III) and the thiol of formula R—SH is conducted in the presence of a base.

15. A composition of matter comprising:
at least one compound of formula (I): 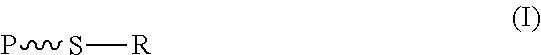
wherein the wavy line indicates the location of the bond between said P and the sulfur atom;
P represents a radical susceptible of generating an odoriferous α,β-unsaturated ketone, aldehyde or carboxylic ester and is represented by the formula 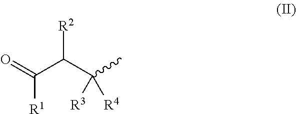
in which the wavy line indicates the location of the bond between said P and S;
R¹ represents a hydrogen atom, a C₁ to C₆ alkoxyl radical or a C₁ to C₁₅ linear, cyclic or branched alkyl, alkenyl or alkadienyl radical, optionally substituted by one to four C₁ to C₄ alkyl groups; and
R², R³ and R⁴ represent independently of each other a hydrogen atom, an aromatic ring, or a C₁ to C₁₅ linear, cyclic or branched alkyl, alkenyl or alkadienyl radical, possibly substituted by C₁ to C₄ alkyl groups; or two, or three, of the groups R¹ to R⁴ are bonded together to form a saturated or unsaturated ring having 5 to 20 carbon atoms and including the carbon atom to which said R¹, R², R³ or R⁴ groups are bonded, this ring being possibly substituted by C₁ to C₈ linear, branched or cyclic alkyl or alkenyl groups;
R represents a linear or branched alkyl group having from 8 to 15 carbon atoms, optionally comprising a carboxylic acid or a C₁₋₈ carboxylic ester functional group which is not directly linked to the sulfur atom,
at most 50 μg/kg of thiol or thioether compounds having less than 6 carbon atoms, and
at most 1 ppm of any compound of formula (I′): 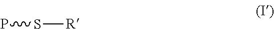
wherein the wavy line indicates the location of the bond between said P and the sulfur atom; P has the same meaning as defined in claims 5 to 14 and R′ represent alkyl group having from 1 to 5 carbon atoms.
